(19) United States
(12) Patent Application Publication
(10) Pub. No.: US 2009/0042969 A1
(43) Pub. Date: Feb. 12, 2009

(54) Use of Specific Trifluoromethyl Ketones for Preventing and Treating Pancreatitis

(76) Inventors: Markus M. Lerch, Greifswald (DE); Julia Mayerle, Greifswald (DE)

Correspondence Address: NIXON & VANDERHYE, PC, 901 NORTH GLEBE ROAD, 11TH FLOOR, ARLINGTON, VA, 22203, US

(21) Appl. No.: 11/990,003

(22) PCT Filed: Aug. 9, 2006
(86) PCT No.: PCT/EP2006/007859

§ 371 Date: Jun. 27, 2008

(30) Foreign Application Priority Data
Aug. 10, 2005 (DE) 10 2005 037 791.2

(51) Int. Cl. A61K 31/40; A61P 29/00

(52) U.S. Cl. 514/423

(57) ABSTRACT

The present invention relates to the use of specific trifluoromethyl ketones for preventing and treating pancreatitis and, more particularly, chronic pancreatitis or chronically recurring pancreatitis.

The present invention relates to the use of specific trifluoromethyl ketones for preventing and treating pancreatitis and, in particular, chronic pancreatitis.

Both chronic and acute pancreatitis are common disorders of the gastrointestinal tract with great socioeconomic importance.

Chronic pancreatitis, with an incidence of 8.2 cases/100 000 population and a prevalence of 27.4 cases, is one of the common disorders of the gastrointestinal tract. The most common cause of chronic pancreatitis is alcohol abuse.

Hereditary pancreatitis is a form of chronic pancreatitis with autosomal dominant inheritance and a phenotypic penetrance of up to 80%. It is initiated in particular by a mutation in the PRSS1 gene which codes for cationic trypsinogen (Withcomb et al., 1996). It is characterized by recurrent episodes of pancreatitis, which usually start in early childhood, and by a usually positive family history, a substantially equal sex distribution and the absence of other disease-associated risk factors.

Chronic pancreatitis is an intermittent, non-infectious inflammation of the pancreas. It may be associated with focal necroses, inflammatory infiltrates, fibrosis of the parenchyma, calculus formation in the ducts and the formation of pseudocysts. In advanced stages there is a global impairment of function with reduced exocrine and endocrine pancreatic function progressing to exocrine and endocrine pancreatic insufficiency (pancreoprivic diabetes mellitus) (Ammann et al., 1984).

The leading symptom of chronic pancreatitis is belt-like upper abdominal pain, weight loss, associated with steatorrhea, and diabetes mellitus. The diagnosis is generally made by imaging methods such as transabdominal ultrasound and ERCP or by investigating pancreatic function.

As causal therapeutic approaches are lacking, the therapy of chronic pancreatitis is limited to controlling symptoms. The aims of treatment of chronic pancreatitis are compensating the exocrine pancreatic insufficiency and thus treating the symptoms of maldigestion, steatorrhea and weight loss, treating the diabetic status and appropriate pain therapy.

Peripherally acting analgesics are recommended for the pain therapy and, in the second stage, can be combined with neuroleptics or tramadol sulfate. The prescription of potent centrally acting opioids is envisaged in the third stage. Insulin therapy should be considered at an early time for the treatment of pancreoprivic diabetes mellitus. Pancreatic enzyme replacement is indicated if there is a weight loss of more than 10% of body weight, steatorrhea with fecal fat excretions of more than 15 g/day, dyspeptic symptoms with severe meteorism or diarrhea. Most enzyme products contain a powdered extract from pig pancreas with the main components lipase, amylase, trypsin and chymotrypsin. The products are administered in the form of microspherically encapsulated formulations. About 30-60% of patients develop complications of their disorder such as strictures of the ductus hepatocholedochus, inflammatory space-occupying lesions, pancreatic pseudocysts or pancreatic duct calculae, which require interventional or surgical therapy.

ERCP (ERCP: endoscopic retrograde cholangiopancreatography) is a demonstration of the biliary tract and of the pancreatic duct system. Such an ERCP is followed in 3-6% of cases by development of pancreatitis. This investigation-associated complication shows a mortality of about 1 per thousand. It has been possible to show that prophylactic treatment with a protease inhibitor which inhibits trypsin, kallikrein and plasmin and which was administered by infusion was able to reduce the damage to the pancreas caused by ERCP (Cavallini et al., 1996).

Chronic recurrent pancreatitis is a form of acute or chronic pancreatitis with recurrent attacks of painful and inflammatory episodes. Possible underlying causes are:

the factors which initiate acute pancreatitis or chronic pancreatitis. The long-term course and the complications which normally occur usually correspond to chronic pancreatitis.

With this background, the object of the present invention is to provide compounds which are suitable for the prevention (prophylaxis) and treatment (therapy) of pancreatitis, especially of chronic pancreatitis, of post-ERCP pancreatitis and of chronic recurrent pancreatitis. A simple form of administration and a reduction of side effects is crucial for this.

The object is achieved according to the invention by the subject matter of claims 1 to 7. It has surprisingly been found that peptidyl trifluoromethyl ketones and their solvates are particularly suitable for the treatment of pancreatitis, especially chronic pancreatitis, chronic recurrent pancreatitis and post-ERCP pancreatitis. An example of a possible solvate is a hydrated form. This may exist for example as geminal diol of the trifluoro ketone group. However, solvates may likewise occur in a form which contains water molecules as part of the crystal lattice.

The invention thus relates to the use of peptidyl trifluoromethyl ketones or their solvates for producing a pharmaceutical composition for preventing and treating pancreatitis.

In a preferred embodiment of the invention, the peptidyl trifluoromethyl ketone is the compound I, i.e. 1-[2-(4-methoxybenzamide)-3-methylbutyryl]-N-[2-methyl-1-(trifluoroacetyl)propyl]pyrrolidine-2-carboxamide of the formula

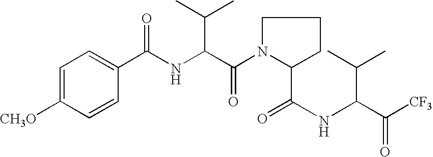

which is used to produce a pharmaceutical composition for preventing and treating pancreatitis.

In a particularly preferred embodiment of the invention, the peptidyl trifluoromethyl ketone is compound II

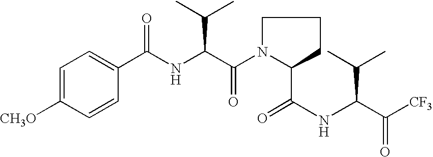

In one embodiment, the pancreatitis is a non-acute pancreatitis, an ERCP-induced pancreatitis or a chronic or chronic recurrent pancreatitis.

Processes for preparing the compounds of the invention are described in the international application WO 95/21855, which is incorporated herein by reference. Thus, for example, various processes for preparing the compound II are described on pages 8-16 of WO 95/21855.

Compound II is the so-called S,S,S-stereoisomer of compound I and is, in one embodiment of the invention, in a form which comprises a proportion not exceeding 10% of the other possible stereoisomers of the compound I. In a preferred embodiment, this proportion does not exceed 5%, and in a particularly preferred embodiment it does not exceed 2%.

WO 95/21855 discloses that compound II can be employed for the treatment of respiratory disorders.

It has surprisingly been found in the context of the present invention that the compounds of the invention are also suitable for a prophylactic treatment of pancreatitis, especially of chronic pancreatitis, chronic recurrent pancreatitis and post-ERCP pancreatitis. Thus, for the first time, active ingredients which can be administered orally and are therefore particularly suitable for prophylaxis are available for treating the aforementioned disorders.

In a preferred embodiment, the compound I, and in a particularly preferred embodiment the compound II, is therefore employed for producing a pharmaceutical composition for the prophylactic treatment of chronic pancreatitis. The pharmaceutical compositions can in this case be administered by intravenous injection or intraperitoneally. In a preferred embodiment, the pharmaceutical compositions are formulated for oral administration. The oral bioavailability makes the pharmaceutical compositions particularly suitable for continuous prophylactic treatment.

For a prophylactic treatment of chronic pancreatitis or of chronic recurrent pancreatitis, the pharmaceutical compositions comprise the corresponding peptidyl trifluoromethyl ketone in an amount of about 10 to 240 mg/kg of body weight if the composition is administered daily.

The pharmaceutical composition can be administered continuously for a prophylactic treatment of chronic pancreatitis or chronic recurrent pancreatitis, in a preferred embodiment the composition being administered orally three times a day, for example in the morning, at midday and in the evening. The biological half-life of the compound in rats, hamsters and dogs is between 0.82 and 2 hours. The intravenous AUC (bioavailability measured as the ratio of intravenous to oral administration as area under the curve (AUC)) [ng*h/ml] is between 1365 and 3179 ng*h/ml (bioavailability measured as ratio of intravenous to oral administration as area under the curve (AUC)). The oral bioavailability is between 355 to 1400 ng*h/ml. The percentage bioavailability was determined to be 26-82%. Oral administration of 10 mg/kg of body weight of the compound II leads to 84% inhibition of neutrophil elastase. To inhibit 50% of the enzyme activity it is necessary to employ 4.9 mg/kg of body weight on oral administration, but only 0.59 mg/kg of body weight on intravenous administration.

In a further embodiment, compound I or compound II is used to produce a pharmaceutical composition for prophylactic treatment before an ERCP investigation, i.e. to prevent post-ERCP pancreatitis.

A wide variety of approaches have been proposed for preventing ERCP-induced pancreatitis, such as, for example, prophylactic administration of aprotinin, glucagon, calcitonin, nifedipine or somatostatin, but all these were unsuccessful. Surprisingly, the compounds of the invention are suitable for effective prophylactic treatment of ERCP-induced pancreatitis.

The pharmaceutical compositions can be administered by infusion before an ERCP. In a preferred embodiment, the pharmaceutical composition is administered orally before an ERCP. The amount of the appropriate compound (preferably compound I or II) can in this connection vary in a range from 10 to 240 mg/kg of body weight. In a preferred embodiment, the amount used is from 30 to 120 mg/kg of body weight. A single administration takes place 30 minutes before the ERCP, either as oral administration or as intravenous administration in a concentration which is 10 times lower.

In a further preferred embodiment of the invention, the uses according to the invention further include the employment of a pharmaceutically suitable carrier material.

The invention is described below by means of examples and figures. The examples are intended to illustrate the invention but are not intended to restrict the invention in any way.

### DESCRIPTION OF THE FIGURES

FIG. 1 shows the inhibitory effect of compound II. Human neutrophil elastase and, to a somewhat smaller extent, also pancreatic elastase is inhibited by compound II with 50% inhibition at 50 nM (neutrophil elastase) and 1 μM (pancreatic elastase).

Trypsin, myeloperoxidase, cathepsin B and L are not significantly influenced by incubation with compound II. Ki of neutrophil elastase 6.7 nM, Ki of pancreatic elastase 200 nM.

FIG. 2 shows the structure of an experiment to investigate the ability of compound II, and to reduce the local and systemic damage during acute pancreatitis.

FIG. 3 shows the assessment of pancreatic edema: FIG. 3A shows the water content of the pancreas. FIG. 3B shows the gross pancreatic findings after supramaximal caerulein stimulation. The gelatinous mass of the pancreas (marked with a circle) which has a water content of 90% is noteworthy.

FIG. 4 shows the measurement of myeloperoxidase activity (MPO) in pancreatic tissue after supramaximal caerulein stimulation. There is a highly significant increase in enzyme activity after four and twelve hours (FIG. 4A). The maximum activity was found after twelve hours. The bars indicate the average values in mU of MPO activity/mg of pancreatic protein ±SEM (SEM: standard deviation of the sample distribution: σₓ=√1/N'(xᵢ−x)2), of five animals per time interval. Western blot analysis shows a continuous rise in leukocyte elastase expression in the pancreas (FIG. 4B). The ultrastructural analysis of the pancreatic tissue of animals which were incubated with caerulein for one hour shows transmigration of neutrophils from vessels into the interstitial space between the pancreatic acinar cells.

FIG. 5 shows light micrographs of the pancreatic tissue (5A).

Quantification of the number of vacuoles shows a highly significant reduction to approximately 15 to 25%.

FIG. 6 shows the serum amylase activity.

FIG. 7 shows light micrographs of the pulmonary tissue (7B). Measurement of the myeloperoxidase activity (MPO) in pancreatic tissue after supramaximal caerulein stimulation in the indicated time intervals shows a highly significant increase in enzyme activity after four and twelve hours (7A). The bars show the average values in mU of MPO activity per mg of lung protein ±SEM of five animals at each time point.

### EXAMPLES

### Materials Used

Caerulein was obtained from Pharmacia, Freiburg, Germany. Human neutrophil elastase was purchased from Calbiochem (San Diego, Calif., U.S.A.). This commercial product was purified from human serum by HPLC (protein concentration >20 units/mg of protein specific activity; in 50 mM sodium acetate pH 5.5 and 200 mM sodium chloride; purity >95%). Human myeloperoxidase was purchased from Calbiochem and purified from blood by HPLC (1 mg/10 ml protein concentration, 150 to 200 units mg protein specific protein activity, in 100 mM sodium chloride, 50 mM sodium acetate, purity >95%). Porcine pancreatic elastase was purchased from Calbiochem and was purified from porcine pancreas. The specific activity was 50 units/mg of protein in 50 mM potassium phosphate buffer, pH 7.0. Cathepsin B was purchased from Calbiochem. This commercial product was purified from human liver by HPLC (1.136 mg/ml protein concentration, 22 units/mg of protein specific activity; in 20 mM sodium acetate (pH 5.0) and 1 mM EDTA; purity >95%). Before use, Cathepsin B was activated with 1 mM dithiotreithol (DTT) on ice for 30 minutes. Cathepsin L was purchased from Calbiochem and prepared from human liver with a concentration of 1584 mU/mg of protein. The substrates used for the analyses were [CBZ-Ile-Pro-Arg]₂-rhodamine 110 and [CBZ-Arg₂]-amino-methylcoumarin (AMC) from Molecular Probes (Eugene, Oreg., U.S.A.), [CBZ-Ala₄]-aminomethylcoumarin (AMC) from Molecular Probes and [CBZ-Phe-Arg]-rhodamine 110 (R110) (Eugene Oreg. U.S.A.). All further chemicals were purchased in the highest purity either from Sigma-Aldrich (Eppelheim, Germany) or Merck (Darmstadt, Germany), Amersham Pharmacia Biotech (Buckinghamshire, UK) or Bio-Rad (Hercules, Calif., U.S.A.).

The animals were bred in the Charles-River breeding laboratories (Sulzbach, Germany). All the experiments were carried out in compliance with the guidelines of the local animal use and animal protection regulations.

### Example 1

### In Vitro Analysis of the Inhibitor Specificity and Capacity

The activity of neutrophil elastase, pancreatic elastase, of trypsin, cathepsin L, cathepsin B, and myeloperoxidase was determined in the absence and presence of various concentrations of compound II (the concentrations were in the range from 1 nM to 1 mM). The trypsin activity (10 mU), myeloperoxidase activity (100 mU) and the neutrophil elastase activity (10 mU) was determined by using the specific fluorogenic substrates [CBZ-Ile-Pro-Arg]₂-rhodamine 110 or [CBZ-Ala₄]-aminomethylcoumarin (AMC) in 100 mM tris-HCl buffer (pH 8.0), which contained 5 mM CaCl₂, 10 μM substrate (final concentration) and 10 mU of bovine trypsin in a volume of 150 μl, at an excitation wavelength of 485 nm and an emission wavelength of 530 nm or an excitation wavelength of 350 nm and an emission wavelength of 460 nm at 37° C. The initial rates of the substrate hydrolysis were measured as arbitrary fluorescence units per minute. The cathepsin B activity (100 mU) and cathepsin L activity (100 mU) were then determined in 0.25 M sodium acetate buffer (pH 5.0), which contained 2 mM EDTA 1 mM DTT, 10 μM of the specific substrate CBZ-[Arg]₂-aminomethylcoumarin or 10 μM [CBZ-Phe-Arg]-rhodamine 110 (final concentration) in a final volume of 150 μl, at an excitation wavelength of 350 nm and an emission wavelength of 460 nm or 485 nm and an emission wavelength of 530 nm at 37° C.

Cleavage of the substrates was measured for 60 minutes in a microplate fluorescence reader (SPECTRAmax GEMINI, Molecular Devices, Sunnywell, Calif., U.S.A.) and compared with the initial activity in the absence of compound II. The results were calculated in percent of the initial enzyme activity (Kukor et al., 2002).

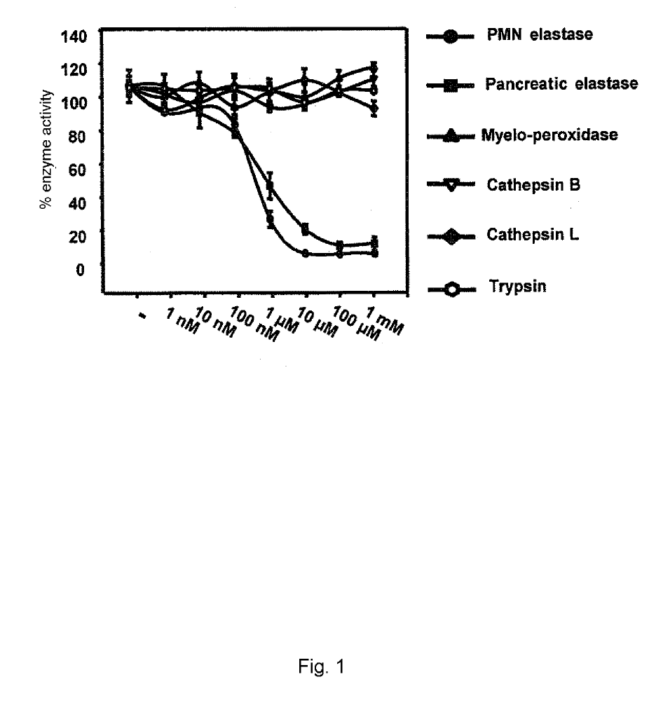

The results are depicted in FIG. 1. Compound II does not inhibit cathepsin B and L, myeloperoxidase and trypsin. The inhibitory activity of compound II for the inhibition of human neutrophil elastase was five times higher by comparison with pancreatic elastase.

### Example 2

### Induction of an Acute Caerulein-Induced Pancreatitis

Male Wistar rats (140 to 250 g) were anesthetized with pentobarbital (30 mg/kg). A cannula was placed in the jugular vein, and a supramaximal concentration of caerulein (10 μg/kg per hour) was administered to the animals by infusion for four and twelve hours. Male Wistar rats (250 to 300 g) received compound II (with a concentration of 240 mg/kg per day) by tube feeding three hours and one hour before induction of the caerulein pancreatitis for four and twelve hours. Animals which received a saline solution as infusion and were fed with compound II through a tube served as control. Each treatment group consisted of five animals. After the exsanguination under ether anesthesia, the pancreas was quickly removed and freed of fat, and tissue blocks were fixed in 5% paraformaldehyde for the histology in paraffin-embedded sections. The largest portion of the pancreas was frozen in liquid nitrogen and stored at −80° C. for later protein analysis and for detection of the enzyme activity.

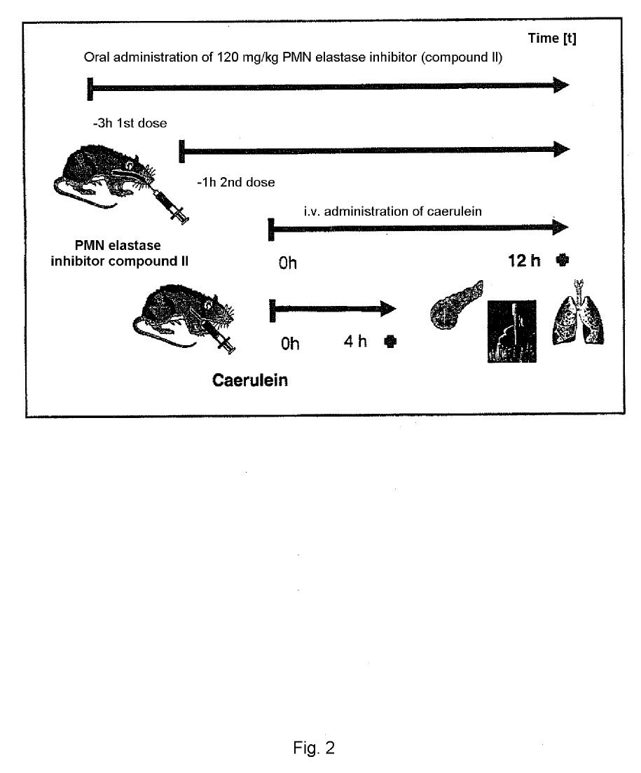

In order to investigate the ability of compound II to reduce the local and systemic damage resulting from pancreatitis, groups each of five male Wistar rats were used as follows: control 1 (vehicle solution), control 2 (compound II), supra-maximal concentration of caerulein (10 μg/kg per hour) for four and twelve hours, supramaximal amount of caerulein+oral feeding of compound II with a concentration of 240 mg/kg per day, with compound II being administered orally by tube feeding in each case three hours and one hour before inducing the caerulein pancreatitis, and being dissolved in 100 μl of polyethylene glycol 400. The animals were sacrificed after four and twelve hours, and pancreatic tissue, serum and lung tissue were collected for further analyses (see FIG. 2).

### Example 3

### Effect of Compound II on the Extent of Pancreatic Edemas

Determination of the pancreatic water content: the water content of the pancreas was determined by comparing the freshly obtained tissue (wet weight) with the weight of the same sample after drying (dry weight). In order to ensure complete drying, the tissue was incubated at 160° C. for 24 hours, a constant weight being reached. The results were represented as percentage of the difference between the wet weight and the dry weight divided by the original wet weight.

Administration of compound II alone did not lead to a significant change in the water content of the pancreas. The animals treated as control 1 showed a water content of approximately 60% which did not differ significantly from the group which was fed orally with compound II (control 2).

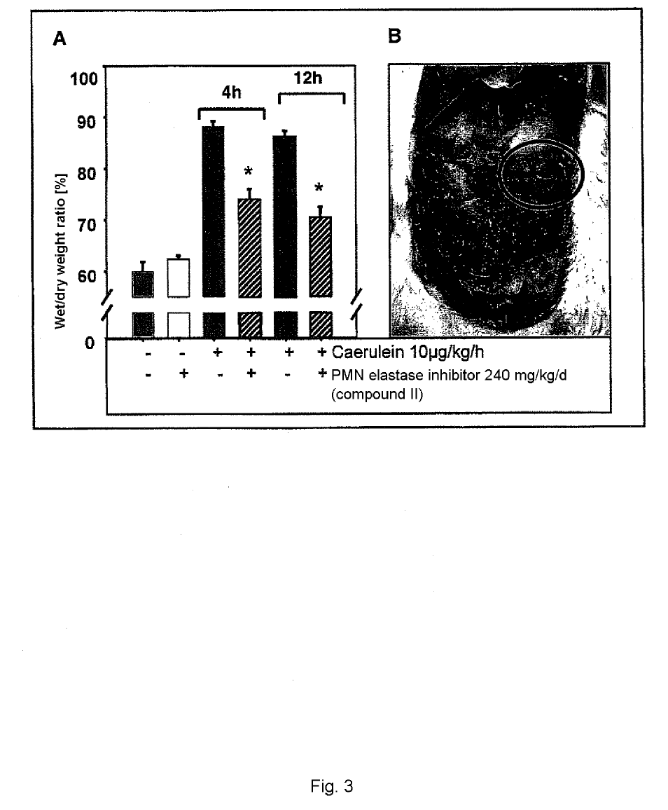

The caerulein infusion induced a pancreatic edema which was reflected by an increase in the water content to up to 90%. Oral administration of compound II showed a significant reduction in the formation of edemas both after four and after twelve hours of starting the caerulein infusion (FIG. 3). The picture on the right in FIG. 3 shows the gross finding in the rat abdomen after induction of edematous pancreatitis.

### Example 4

### Determination of Myeloperoxidase Activity in Pancreatic Homogenates

To measure the myeloperoxidase (MPO) activity, the tissue was thawed and homogenized on ice in 20 mM potassium phosphate buffer (pH 7.4) and centrifuged at 20 000 g at 4° C. for 10 minutes. The pellet was resuspended in 50 nM potassium phosphate buffer (pH 6.0) which contained 0.5% cethyltrimethylammonium bromide. The suspension was frozen and rethawed four times, treated once with sound waves at an output level of 30% for 10 seconds and centrifuged at 20 000 g at 4° C. for 10 minutes. The MPO activity was investigated after mixing 50 μl of supernatant in 200 μl of a 50 mM potassium phosphate buffer (pH 6.0) which contained 0.53 mm O-dianisidine and 0.15 mU H₂O₂.

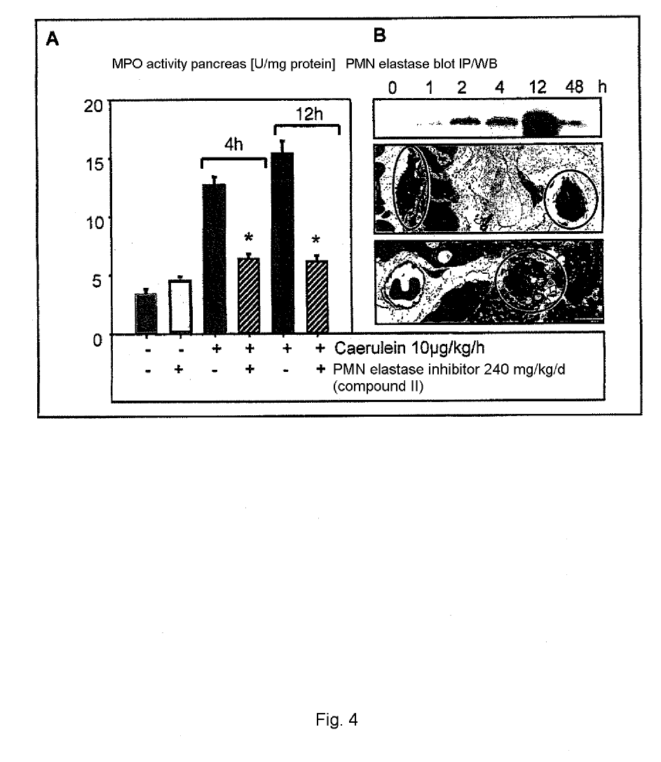

The original increase in the absorption at 450 nm was measured at room temperature using a Dynatech MR 5000 ELISA reader. The results have been represented as units of MPO, where one unit oxidizes 1 μM H₂O₂ per minute per mg of pancreatic protein. The bars show average values in mU of MPO activity/mg of pancreatic protein of five animals per time point (see FIG. 4).

The intention of measuring the myeloperoxidase activity was to determine the number of neutrophils which penetrate into the pancreatic tissue. A direct interaction of compound II with MPO was excluded in in vitro experiments in order to ensure that the reduced MPO levels correlated with a diminished neutrophil infiltration and were not caused by the inhibition of MPO. The animals treated with compound II showed more than 50% reduction in MPO activity in pancreatic tissue.

Since the MPO activity in pancreatic tissue only represents an indirect determination of the presence of neutrophils, immuno-precipitation experiments were likewise carried out for the time course of the caerulein pancreatitis up to 48 hours, and the samples were analyzed for expression of neutrophil elastase by Western blotting.

### Immunoprecipitation and Immunoblotting:

The pancreatic tissue was homogenized in ice-cold PBS or Triton X 100 lysis buffer which contained protease inhibitors (1 ml/mg of tissue), 10 μg/ml aprotinin, 10 μg/ml leupeptin, 0.01 M sodium pyrophosphate, 0.1 M sodium fluoride, 1 mM dihydrogen peroxide, 1 mM L-phenylmethylsulfonyl fluoride PMSF and 0.02% soybean trypsin inhibitor), with a Dounce S glass homogenizer (Braun-Melsungen) for Western blots or without inhibitors for detecting the enzymatic activities. The protein concentration was determined by modified Bradford assay (Bio-Rad), and identical amounts of protein were employed in each of the subsequent experiments. For the immunoprecipitation, a mixture of protein A and G-sepharose (Amersham Pharmacia Biotech) was preincubated with antibody in 20 mM HEPES pH 7.4. The lysates were prepurified with rat non-immune serum, added to the coupled antibodies and incubated at 4° C. on a rotating disk for one hour. The precipitates were washed with HNTG (HNTG: HEPES-NaCl-Triton-glycerol buffer) and boiled in 2×SDS sample buffer for five minutes. SDS polyacrylamide gel electrophoresis was carried out in a discontinuous buffer system, and the gels were blotted onto nitrocellulose membranes (Highbond C, Amersham Pharmacia Biotech). After blocking in NET-gelatin 0.2% (NET: sodium-EDTA-tris buffer) overnight, the immunoblot analysis was carried out, followed by a chemoluminescence detection (Amersham Pharmacia Biotech), using horseradish peroxidase coupled to a sheep anti-mouse IgG (Amersham Pharmacia) or goat anti-rabbit IgG (Amersham Pharmacia Biotech).

Neutrophil elastase expression showed its maximum after twelve hours, but had a significant increase even one hour after the start of the pancreatitis. Likewise one hour after the start of the caerulein pancreatitis it was possible to detect transmigration of neutrophils from small vessels into the interstitial space of the pancreas, as demonstrated by the electron microscopy (see FIG. 4).

For the electron microscopy, small blocks (2 mm in diameter) of the pancreatic tissue of the rat pancreas after the supramaximal caerulein infusion and of control animal were fixed in 2% formaldehyde/2% glutaraldehyde, embedded in Epon, and osmium-, uranyl- and lead-contrasted thin sections were employed for the electron microscopy. Ultrathin sections (60 nm) were prepared with the aid of a Leica ultramicrotome. The samples were examined in a Philips 400 electron microscope and photographed at a magnification of 30 000×.

### Example 5

### Effect of Compound II on Morphological Damage

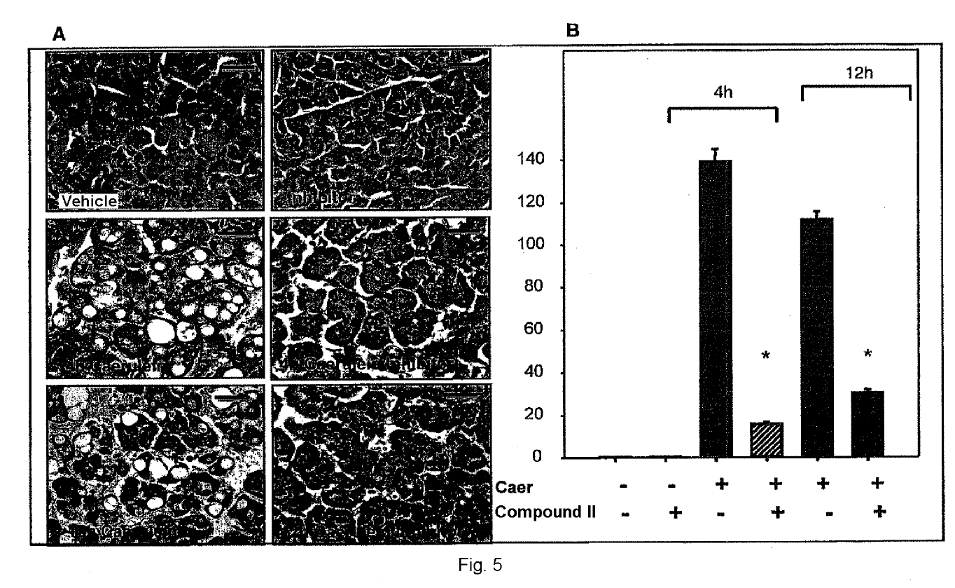

The supramaximal stimulation with caerulein led to an interstitial edema and to the formation of large intracellular vacuoles in pancreatic acinar cells. Administration of compound II did not lead to any morphological changes like those observed in acute pancreatitis. Administration of compound II together with caerulein prevented the development of the interstitial edema and the intracellular vacuolization. The vacuoles which were still detectable were very much smaller, and the number of infiltrating inflammatory cells was greatly reduced. Quantification of the number of vacuoles in the various treatment groups showed a highly significant reduction to approximately 15 to 25% of the vacuoles found with untreated pancreatitis (see FIG. 5).

### Example 6

### Effect of Compound II on the Hyperamylasemia

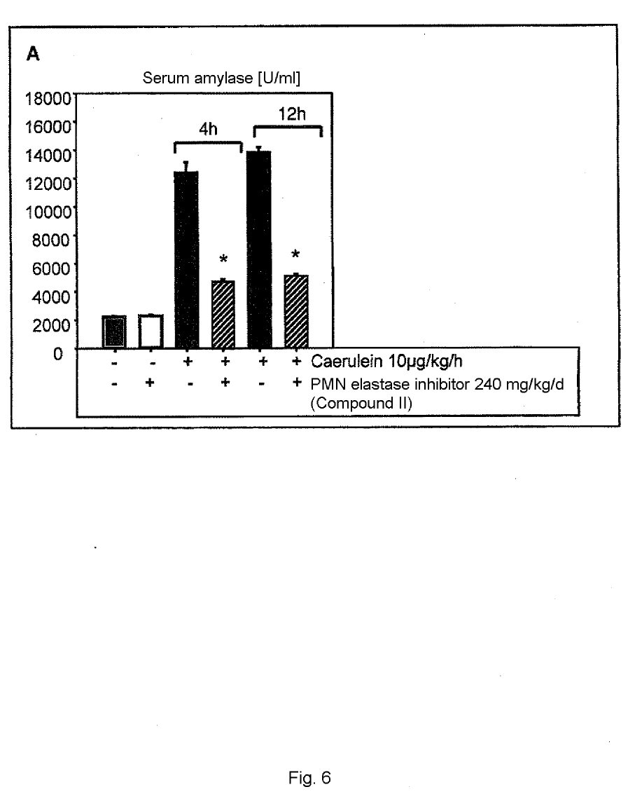

As a result of cell damage, an increased amylase activity in serum can be detected during the course of caerulein-induced pancreatitis even one hour after the start of the pancreatitis. The extent of the pancreatic azinar cell destruction correlates with the extent of the amylase activity in serum. It was shown that the amylase activity detectable in the serum of the animals after four and twelve hours of pancreatitis was significantly reduced by oral administration of compound II (see FIG. 6).

### Example 7

### Effect of Compound II on Myeloperoxidase Activity in the Lung During Pancreatitis

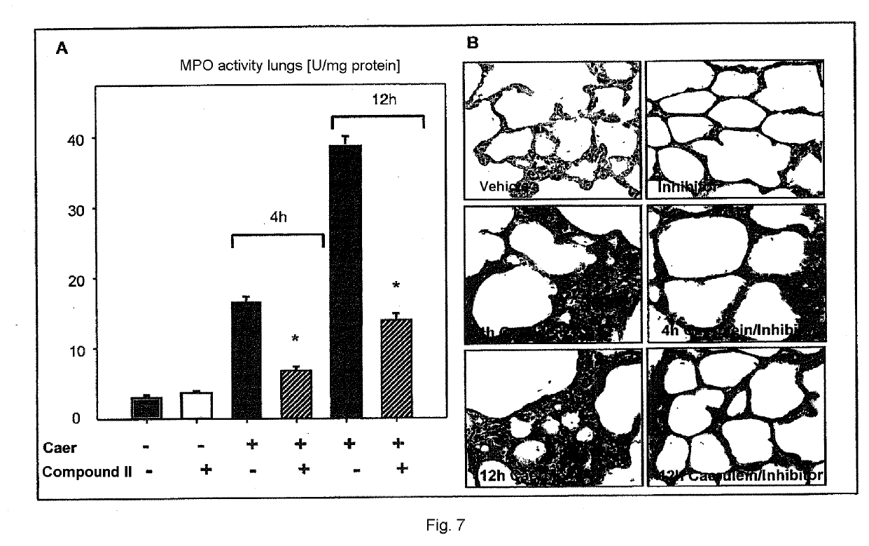

Pancreatitis influences not only the pancreas but also leads to a systemic inflammatory response which affects other organs such as the kidneys or the lung. In order to investigate whether compound II plays a role in influencing the extra-pancreaic effects of the course of the disease, the myeloperoxidase activity in lung tissue was investigated in order to assess the extent of leukocyte infiltration, and to analyze the morphological differences in the lungs of pancreatitis animals and untreated controls. The morphological differences in the lung tissues during caerulein-induced pancreatitis consisted of accumulations of alveolar fluid and a progressive thickening, hyperemia and neutrophil infiltration of the intraalveolar tissue. All these events were reduced by administering compound II. The animal group treated with compound II showed a significant reduction in MPO activity, which correlated with a reduced number of neutrophils infiltrating the lung tissue. The observed effect was greater twelve hours after onset of the pancreatitis than after four hours. This observation is consistent with the fact that the systemic inflammatory response reaches it peak after twelve hours of the course of an acute pancreatitis. These data clearly show that inhibition of neutrophil elastase by an orally administered active ingredient not only reduces local pancreatic damage but likewise influences the systemic inflammatory response during the pancreatitis (see FIG. 7).

### REFERENCES

Ammann R W, Akrovbiantz A, Largiader F, Schueler G. Course and outcome of chronic pancreatitis. Longitudinal study of a mixed medicalsurgical series of 245 patients. Gastroenterology 1984; 86:820-828.
Cavallini G, Tittobello A, Frulloni L, Masci E, Mariani A, Di Francesco V. Gabexate for the prevention of pancreatic damage related to endoscopic retrograde cholangiopancreatography. The New England J. of Med. 1996; 335(13): 919-923.
Dominguez-Munoz J E, Carballo F, Garcia M J, de Diego J M, Rabago L, Simon M A, de la Morena J. Clinical usefulness of polymorphonuclear elastase in predicting the severity of acute pancreatitis: results of a multicentre study. Br. J. Surg 1991; 78:1230-1234.
Geokas M C, Murphy R, McKenna R D. The role of elastase in acute pancreatitis. I. Intrapancreatic elastolytic activity in bile-induced acute pancreatitis in dogs. Arch. Pathol. 1968; 86:117-126.
Kukor Z, Mayerle J, Krüger B, Toth M, Steed P, Halangk W, Lerch M M, Sahin-Toth M. Presence of cathepsin B in the human pancreatic secretory pathway and ist role in trypsinogen activation during hereditary pancreatitis. J. Biol. Chem. 2002; 277:21389-21396.
Withcomb D C, Gorry M C, Preston R A, Furey W, Sossenheimer M J, Ulrich C D, Martin S P, Gates L K, Amann S T, Toskes P P, Liddle R, McGrath K, Uomo G, Post J C, Ehrlich G D. Hereditary pancreatitis is caused by a mutation in the cationic trypsinogen gene. Nat. Genet. 1996; 14:141-145.

1. The use of a peptidyl trifluoromethyl ketone or its solvate for preventing and treating pancreatitis.

2. The use as claimed in claim 1, characterized in that the peptidyl trifluoromethyl ketone has the following structure:

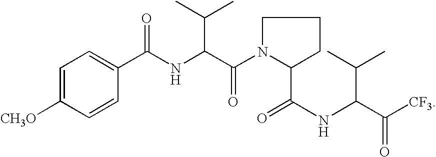

3. The use as claimed in claim 2, characterized in that the peptidyl trifluoromethyl ketone is the following stereoisomer:

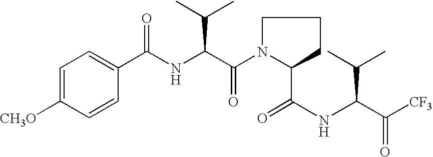

4. The use as claimed in claim 1, characterized in that the pancreatitis is a chronic or chronic recurrent pancreatitis.

5. The use as claimed in claim 1, characterized in that the pancreatitis is a post-ERCP pancreatitis.

6. The use as claimed in claim 1, characterized in that the peptidyl trifluoromethyl ketone is administered orally.

7. The use as claimed in claim 1, characterized in that the use includes the employment of a pharmaceutically suitable carrier material.
